(19) United States
(12) Patent Application Publication
(10) Pub. No.: US 2026/0364126 A1
(43) Pub. Date: Nov. 19, 2026

(54) BACTEROIDES FAECICHINCHILLAE AND ITS USE IN THE TREATMENT OF METABOLIC DISORDERS

(71) Applicants: INSTITUT NATIONAL DE LA SANTÉ ET DE LA RECHERCHE MÉDICALE, Paris (FR); INSTITUT NATIONAL DE RECHERCHE POUR L'AGRICULTURE, L'ALIMENTATION ET L'ENVIRONNEMENT (INRAE), Paris (FR); UNIVERSITÉ CLAUDE BERNARD - LYON 1, Villeurbanne (FR)

(72) Inventors: Hubert VIDAL, Oullins (FR); Annick BERNALIER DONADILLE, Saint Genes Champanelle (FR); Murielle GODET, Pierre-Bénite Cedex (FR); Eve DELMAS, Saint Genes Champanelle (FR)

(21) Appl. No.: 19/133,412

(22) PCT Filed: Nov. 30, 2023
(86) PCT No.: PCT/EP2023/083669

§ 371 Date: May 28, 2025

(30) Foreign Application Priority Data
Dec. 1, 2022 (EP) 22306772.9

(51) Int. Cl. A61K 35/741; A61K 31/155; A61P 3/10

(52) U.S. Cl. CPC A61K 35/741; A61K 31/155; A61P 3/10

(57) ABSTRACT

The invention relates to the field of probiotic and in particular to bacteria of the species Bacteroides faecichinchilleae and its use for treating metabolic disorders. Performing an original screening test, the present inventors identified a novel species of commensal bacteria, Bacteroides faecichinchilleae, which possesses the ability to improve glucose tolerance in a preclinical model of diabetes, with a unique mechanism of action, closely related to the mechanism of anti-diabetic drug metformin such as inhibition of FXR activity in the small intestine (through modifications of intestinal bile acid profile) and reduction of ceramide levels in the liver (especially C16-ceramides). Thus this study opens the way to safe use of Bacteroides faecichinchilleae and accordingly the present invention provides an Bacteroides faecichinchilleae and its use as a drug or probiotic and more particularly for use in the treatment of metabolic disorders such as type 2 diabetes, alone or in combination with classical anti-diabetic drug metformin.

### FIELD OF THE INVENTION

The invention generally relates to the field of probiotics and especially regarding bacteria of the species Bacteroides faecichinchilleae and its use for treating metabolic disorders.

### BACKGROUND OF THE INVENTION

The rising prevalence of metabolic diseases, including type 2 diabetes and obesity, is becoming a worldwide concern. These pathologies are associated with insulin resistance, low-grade inflammation, dyslipidemia and non-alcoholic fatty liver diseases, which are major risk factors for cardio-metabolic disorders and also various cancers (1). Qualitative and quantitative alterations of the gut microbiota, referred to as dysbiosis, have been clearly demonstrated to be important hallmarks of these common metabolic diseases, both in humans and in animal models (1-6). The transfer of microbiota from obese mice conferring the obesity phenotype to axenic mice has established a causal link between dysbiosis and obesity (7). Regarding type 2 diabetes, although there are not optimal rodent models that perfectly reproduce human pathology, it can be mimicked using animals fed a high-calorie diet and by studying insulin resistance, glucose intolerance and inflammation (8). These nutritional models are associated with gut microbiota dysbiosis, characterized by a decrease in Bacteroidetes and an increase in Firmicutes abundances (2, 6, 9). Also supporting a role of gut microbiota in type 2 diabetes, it was found that metformin, the main pharmacological drug used for the treatment of diabetes, modifies the composition of the intestinal microbiota both in mice and in humans, and that this modification is required for its beneficial action (10,11).

The discovery of the involvement of the intestinal microbiota in metabolic diseases has logically boosted research efforts to find solutions to correct dysbiosis and/or restore the functionalities of the microbiota. One of the possible strategies is the use of probiotics, “live microorganisms which confer a health benefit on the host when they are administered in adequate quantities”. The most commonly used probiotics are strains of Lactobacilli and Bifidobacteria and a number of experimental data suggest that these classical probiotics could be useful in the management of metabolic diseases and positive effects on glucose tolerance and insulin sensitivity have been evidenced in mouse models (12-16), However, the effects in diabetic patients are much less significant (17,18) and recent meta-analyses clearly show that currently available probiotics are not very effective in type 2 diabetes (19,20). Alongside these classical probiotic strains, interest has also focused in recent years on the potential of “next generation probiotics” in metabolic diseases. Akkermansia muciniphila, a commensal species abundant in the intestinal tract and greatly reduced in animal models of obesity and diabetes, is the first next-generation bacterium with health effects in obesity to be approved by the European Food Safety Authority (EFSA) as a safe food ingredient. Interestingly, A. muciniphila abundance is boosted by metformin treatment in rodents (11) and its supplementation improves metabolic disturbances in mice (21) and in humans (22). Several other potential next-generation probiotics are currently under investigation with results published for the moment only in pre-clinical models but they are not specifically focused on the treatment of type 2 diabetes (23). Therefore, there is a need for new probiotics in order to treat metabolic disorders such as type 2 diabetes, and that can be used either alone or in combination with current antidiabetic drugs such as metformin (24).

### SUMMARY OF THE INVENTION

The invention is based on the study of the mechanisms implicated in the beneficial therapeutic properties of bacteria of the species Bacteroides faecichinchilleae, in particular the strain B. faecichinchilleae ST37, on metabolic disturbances in a mouse pre-clinical model of diabetes.

Thus, in a first aspect, the present invention provides a bacteria of the species Bacteroides faecichinchilleae for use as a drug or a probiotics.

A second object of the invention relates to a bacteria of the species Bacteroides faecichinchilleae, for use in the treatment of metabolic disorders. A third object of the invention relates to a pharmaceutical composition comprising a bacteria of the species Bacteroides faecichinchilleae and metformin.

Another object of the invention relates to a pharmaceutical composition comprising a bacteria of the species Bacteroides faecichinchilleae and metformin for simultaneous or sequential use in preventing or treating metabolic disorders.

In a particular embodiment, a metabolic disorder is selected from the list consisting of excess weight, obesity, hepatic steatosis or fatty liver, dyslipidemia and, in particular, hypercholesterolemia and/or hypertriglyceridemia; hyperglycemia, insulin resistance and diabetes, preferably Type 2 diabetes mellitus and gestational diabetes; metabolic syndrome, hypertension and cardiovascular diseases.

### DETAILED DESCRIPTION OF THE INVENTION:

### Bacteroides faecichinchilleae and Use for Treatment

In the present invention, inventors developed a full research program to identify, select and validate novel bacterial strains able to improve metabolic alterations in type 2 diabetes. This program was based on 3 major items: 1) a bioinformatic search of potential candidates using published and in-house metagenomic data from diabetic humans and rodents treated with metformin, 2) a first round of screening and qualification using a mice model of high-fat high-sucrose (HFS) diet for 8 days with comparison with the effect of metformin and 3) the validation of the best candidate in a model of diabetic mice (14 weeks of HFS diet) and the characterization of its mechanism of action.

Therefore, this study allowed the inventors, among the tested candidates, to identify the species Bacteroides faecichinchilleae as commensal bacteria able to reproduce some of the effect of metformin in HFS-fed mice. The work was conducted with the Bacteroides faecichinchilleae strain ST37, named BAfa hereafter for simplification. A first set of experiments (see FIG. 1) showed that the expression in the duodenum of the mice of metabolic markers such as Glut5 (which mediates absorption of fructose) and Glut2 (main transporter of glucose), which are significantly increased by HFS diet, are restored at their normal level by metformin as well as by BAfa supplementation during 8 days. Similarly, HFS diet-induced altered levels of expression of the incretin hormones glucagon (Gcg) and gastric inhibitory polypeptide (Gip), which are major actors of insulin secretion in response to glucose load, are also restored at their normal level by metformin and by BAfa supplementation (FIG. 1). Then, experiments in animals fed HFS diet for 14 weeks show that 3 weeks of treatment with live BAfa (10⁹ cfu/day) allows significant improvement of glucose tolerance using intraperitoneal glucose tolerance tests (iGTT), with effects comparable to Akkermansia muciniphila supplementation (10⁹ cfu/day) (see FIG. 2). Although the effect of BAfa was less important than the effect of metformin used at maximal dosage (200 mg/kg/day, for 3 weeks) (see FIG. 3), the results of these experiments (FIGS. 2 and 3) clearly demonstrate that BAfa supplementation improves glucose tolerance in a preclinical model of diabetes. To further document the mechanisms of action contributing to the beneficial effect of BAfa, inventors investigated the impact of the treatment on FXR/FGF15 pathway in the ileum. The farnesoid X receptor (FXR), plays a pivotal role in regulating bile acid, lipid and glucose metabolism, but also inflammation and intestinal barrier function (25). In the ileum, FXR pathway controls the gene expression of Fgf15 (fibroblast growth factor 15 in rodent and FGF19 in humans), which recently emerged as an important regulator of glucose and lipid metabolism in addition to its role in the control of bile acid synthesis in the liver (26, 27). Inventors found that supplementation of HFS mice with BAfa, but not with A. muciniphila, results in a strong inhibition of the expression of the target genes of FXR in the ileum, namely Fgf15, Shp/Nrob2 (small heterodimer partner 1) and Fabp6 (Fatty acid binding protein 6, coding for the ileal intracellular bile acid transporter) (see FIG. 4). These results indicated thus that BAfa supplementation is associated with an inhibition of FXR signaling in the ileum. To determine the causes of this inhibition, bile acid profiling was performed in the ceacum of the treated mice. Bile acids are natural ligands of FXR, with CDCA (chenodeoxycholic acid) and DCA (deoxycholic acid) being the more potent agonists, whereas UDCA (ursodeoxycholic acid) and B-MCA (muricholic acid) are antagonists of FXR. FIG. 5 shows that BAfa supplementation for 4 weeks in diabetic mice, and not A. muciniphila, changes bile acid profile in the gut with a decrease in the amount of secondary bile acids and a reduction in the amount of FXR agonists leading to a lower FXR agonists on FXR antagonists ratio, likely contributing to the observed inhibition of FXR activity in the ileum. Interestingly, inhibition of FXR in the intestinal tract has been recently proposed as a possible mechanism to explain the antidiabetic action of metformin in type 2 diabetic patients (29). Furthermore, other investigators have recently proposed that the beneficial effect of FXR inhibition in the ileum is related to a reduction of the production of ceramides and their accumulation in the liver (30,31). Ceramides are major contributors to insulin resistance, inflammation, hepatic steatosis and metabolic disorders (32,33). Inventors therefore quantified the different ceramides species in the liver of the treated mice. FIG. 6 clearly shown that 4 weeks treatment with BAfa was able to significantly reduce the amount of various ceramide species in the liver of the HFS fed mice, especially C16- and C18-ceramides known to be strongly linked to insulin resistance and to inhibition of insulin signalling and action (33). Furthermore, it was reported that inhibition of ceramide synthesis using pharmacological inhibitors (such a myriacin), leading to an about 50% reduction of C-16 ceramide concentration in the liver, correlates with improvement of insulin action and steatosis in obese mice (34). In the inventor experiments, similar inhibition of C16-ceramide level was observed in mouse liver after 4 weeks of BAfa supplementation (FIG. 6).

Consequently, the Bacteroides faecichinchilleae strain ST37, according to the invention, trigger an efficient response against metabolic alterations, with distinct mechanism of action than other species already proposed for the treatment of metabolic disorder (such as Akkermansia muciniphila) or other Bacteroides previously evaluated in pre-clinical mouse models, such as Bacteroides uniformis which seems to act preferentially on intestinal immune system (35). Globally, the in vivo results obtained by the inventors in a mouse model of diabetes and metabolic disease indicate that the species Bacteroides faecichinchilleae is particularly suited for use in these pathologies, in comparison to the rest of the bacterial previously proposed with this aim, particularly A. muciniphila that do not have similar properties, especially the inhibition of FXR activity in the small intestine.

Accordingly as a first aspect, the present invention provides a bacteria of the species Bacteroides faecichinchilleae for use as a probiotic or as a drug.

The term “probiotic” has its general meaning in the art and refers to a live microorganism that, when administered in adequate amounts, confers a health benefit on the host” (see: Clinical Infectious Diseases, Volume 46, Issue Supplement_2, 1 Feb. 2008, Pages S58-S61, https://doi.org/10.1086/523341).

The term “Bacteroides” a genus of Gram-negative, obligate anaerobic bacteria. Bacteroides species are non endospore-forming bacilli, and may be either motile or nonmotile, depending on the species. The DNA base composition is 40-48% GC. Unusual in bacterial organisms, Bacteroides membranes contain sphingolipids. They also contain meso-diaminopimelic acid in their peptidoglycan layer. Bacteroides species are normally mutualistic, making up the most substantial portion of the mammalian gastrointestinal microbiota, where they play a fundamental role in processing of complex molecules to simpler ones in the host intestine (Wexler H M (2007). Clinical Microbiology Reviews. 20 (4): 593-621). As many as 1010-1011 cells per gram of human feces have been reported, they can use simple sugars when available; however, the main sources of energy for Bacteroides species in the gut are complex host-derived and plant glycans (Martens E C, et al (2008) Cell Host & Microbe. 4 (5): 447-57).

The term “Bacteroides faecichinchilleae” (also called Bacteroides faecichinchilleae ST37 strain or JCM 17102) means is an anaerobe, mesophilic, Gram-negative bacterium that was initially isolated from chinchilla feces (as described in Kitahara M. et al International Journal of Systematic and Evolutionary Microbiology (2012), 62, 1145-1150). Bacteroides faecichinchillae JCM 17102 is also referenced in NCBI Taxonomy ID: 1236513 (NCBI: txid1236513) in BacDive ID: 22936. The Genbank accession numbers are: 16S rRNA gene: AB574480 and whole genome shotgun sequence: FQVD00000000. The strain ST37 of Bacteroides faecichinchilleae may also be obtained from the German Collection of Microorganisms and Cell Cultures (DSMZ 26883) (25) and cultured under anaerobic condition.

In a particular embodiment, The term “Bacteroides faecichinchilleae” also encompass its cellular components, metabolites and secreted molecules, and to compositions which, comprising at least one of the foregoing products,

Accordingly another aspect of the present invention relates to cellular components, metabolites, secreted molecules or any combination thereof, obtained from the strain of the invention or from a mixture of microorganisms comprising at least one strain of the invention.

The cellular components of the bacterium may include the components of the cell wall (such as, but not limited to, peptidoglican), nucleic acids, membrane components, or others such as proteins, lipids and carbohydrates and combinations thereof, such as lipoproteins, glycolipids or glicoproteins. The metabolites include any molecule produced or modified by the bacterium as a consequence of their metabolic activity during growth, their use in technological processes (for example, but not limited to, food or drug elaboration processes) during product storage or during gastrointestinal transit. Examples of these metabolites are, but not limited to, organic and inorganic acids, proteins, peptides, amino acids, enzymes, lipids, carbohydrates, lipoproteins, glycolipids, glycoproteins, vitamins, salts, metals or nucleic acids. The secreted molecules include any molecule exported or released by the bacterium during growth thereof, its use in technological processes (for example, preparation of food or drugs), product storage or gastrointestinal transit. Examples of these molecules include, but not limited to, organic and inorganic acids, proteins, peptides, amino acids, enzymes, lipids, carbohydrates, lipoproteins, glycolipids, glycoproteins, vitamins, salts, metals or nucleic acids.

Thus typically, Bacteroides faecichinchilleae according to the invention has the capacity (i) to improve glucose tolerance, and/or (ii) to inhibit of FXR activity in the small intestine (through modifications of intestinal bile acid profile) and/or (iii) to reduce ceramide levels in the liver (especially C16-ceramides).

The skilled in the art can easily determine whether Bacteroides faecichinchilleae, or the cellular components, metabolites, molecules secreted by said strains and combinations thereof, according to the invention is biologically active. For example, the capacity to improve glucose tolerance can be determined by any routine test well known by the man skills in the art: intraperitoneal glucose tolerance tests (iGTT) as described in experimental section. Typically the glucose tolerance test measures the clearance of an intraperitoneally injected glucose load from the body. It is used to detect disturbances in glucose metabolism that can be linked to human conditions such as diabetes or metabolic syndrome. Animals are fasted several hours before the test and blood glucose levels are determined before a solution of glucose is administered by intra-peritoneal (IP) injection. Subsequently, the blood glucose level is measured at different time points during the following 2 hours (see www.mmpc.org/shared/document.aspx?id=238&docType=Protocol)

The inhibition of intestinal FXR activity may be assessed by any routine test well known by the man skills in the art (such as described in experimental section), for example by monitoring the reduction of the expression levels of FXR target genes in the ileum, namely Fgf15, Shp/ Nrob2 (small heterodimer partner 1) and/or Fabp6 (Fatty acid binding protein 6, coding for the ileal intracellular bile acid transporter), as also described by other investigators (see 30) . Inhibition of FXR activity can also be assessed by monitoring changes in bile acid composition in the intestinal tract (for example in the caecum) using classical methodologies (such as described in experimental section), and for example by monitoring the ratio between bile acids that are agonists of FXR (such as DCA and HDCA) and those that are antagonists of FXR (such as UDCA, β-MCA) (see 28,30).

The reduction of hepatic ceramide concentration, especially the deleterious C16- and C18-ceramides, activity can be assessed by any routine test well known by the man skills in the art (such as described in experimental section (material and method“ Ceramide profiling in liver”: Concentrations ceramide molecular species is determined according to the method by Kyrklund (40)). It can be indicated that the use of ceramide synthesis inhibitor (such as myriacin) has been already documented as beneficial for insulin resistance and liver steatosis (34).

As used herein, a “biologically active” Bacteroides faecichinchilleae according to the invention refers to a Bacteroides faecichinchilleae strain, or the cellular components, metabolites, molecules secreted by said strain and combinations thereof, exhibiting at least one of the biological activities of the wild-type Bacteroides faecichinchilleae. The biologically active Bacteroides faecichinchilleae according to the invention may for example be characterized in that it is capable of i) improving glucose tolerance and/or ii) inhibiting of intestinal FXR activity and/or iii) reducing hepatic C16 and C-18 ceramide concentrations.

A second object of the invention relates to the Bacteroides faecichinchilleae bacterium, as defined above for use in the treatment or prevention of metabolic disorders.

The term “metabolic disorders” means a disorder that negatively alters the body's processing and distribution of macronutrients, such as proteins, fats, and carbohydrates. Metabolic disorders can happen when abnormal chemical reactions in the body alter the normal metabolic process. It can also be defined as inherited single gene anomaly, most of which are autosomal recessive. A “metabolic disorder” according to the present invention means metabolic alterations such as glucose or lipid metabolic disorder and directly associated with excess weight, obesity, hepatic steatosis or fatty liver, dyslipidemia and, in particular, hypercholesterolemia and/or hypertriglyceridemia; hyperglycemia, insulin resistance and diabetes, preferably type 2 diabetes mellitus and gestational diabetes; metabolic syndrome. hypertension and cardiovascular diseases.

The present disclosure demonstrates how a strain of the species Bacteroides faecichinchilleae (ST37 strain) may be used to treat and/or to prevent other lipid and glucose metabolism alterations, not necessarily associated with excess weight or obesity, such adipocyte hypertrophy; hepatic steatosis or fatty liver, dyslipidemia (i.e. hypertriglyceridemia and/or hypercholesterolemia), hypertension, cardiovascular diseases, hyperglycemia, insulin resistance and/or diabetes (for example, gestational diabetes or type 2 diabetes mellitus); or metabolic syndrome. Likewise, it was observed that said strain of Bacteroides faecichinchilleae can be used to improve the function of the immune system, for example, reducing inflammation (through the beneficial effect of FXR inhibition and reduction of ceramide species) caused by the previously described chronic metabolic alterations.

Accordingly, another object of the present invention relates to a method of treating or preventing metabolic disorder in a subject thereof, the method comprising administering the subject a therapeutically effective amount of Bacteroides faecichinchilleae of the invention, and/or the cellular components, metabolites, molecules secreted by said strains and combinations thereof.

Treatment may be for any purpose, including the therapeutic treatment of subjects suffering from metabolic disorder, as well as the prophylactic treatment of subjects who do not suffer from metabolic disorders (e.g., subjects identified as being at high risk for metabolic disorders). As used herein, the terms “treatment,” “treat,” and “treating” refer to reversing, alleviating, inhibiting the progress of a disease or disorder as described herein (i.e. metabolic disorders), or delaying, eliminating or reducing the incidence or onset of a disorder or disease as described herein, as compared to that which would occur in the absence of the measure taken. The terms “prophylaxis” or “prophylactic use” and “prophylactic treatment” as used herein, refer to any medical or public health procedure whose purpose is to prevent the disease herein disclosed (i.e. metabolic disorder). As used herein, the terms “prevent”, “prevention” and “preventing” refer to the reduction in the risk of acquiring or developing a given condition (i.e. metabolic disorder), or the reduction or inhibition of the recurrence or said condition (i.e. metabolic disorder) in a subject who is not ill, but who has been or may be near a subject with the condition (i.e. metabolic disorder)

In a specific embodiment Bacteroides faecichinchilleae may be used to treat or prevent metabolic disorder such as obesity, hepatic steatosis or fatty liver, dyslipidemia and, in particular, hypercholesterolemia and/or hypertriglyceridemia; hyperglycemia, insulin resistance and diabetes, preferably type 2 diabetes mellitus and gestational diabetes; metabolic syndrome, hypertension and cardiovascular diseases.

In a more specific embodiment Bacteroides faecichinchilleae species of the invention, or the cellular components, metabolites, molecules secreted by said strains and combinations thereof, may be used to treat metabolic disorder such as insulin resistance and type 2 diabetes mellitus.

As used herein, the term “type 2 diabetes mellitus” is a disease known as adult-onset diabetes, is a form of diabetes mellitus that is characterized by high blood sugar, insulin resistance, and relative lack of insulin. Long-term complications from high blood sugar include heart disease, strokes, diabetic retinopathy which can result in blindness, kidney failure, and poor blood flow in the limbs which may lead to amputations (“Diabetes Fact sheet No 312”. World Health Organization. August 2011). The sudden onset of hyperosmolar hyperglycemic state may occur; however, ketoacidosis is uncommon (Pasquel F J, et al (2014). Diabetes Care. 37 (11 ): 3124-31). Type 2 diabetes makes up about 90% of cases of diabetes, with the other 10% due primarily to type 1 diabetes and gestational diabetes (“Diabetes Fact sheet No 312”. World Health Organization. August 2011). In type 1 diabetes there is a lower total level of insulin to control blood glucose, due to an autoimmune induced loss of insulin-producing beta cells in the pancreas (Mackay I, Rose N, eds. (2014). The Autoimmune Diseases. Academic Press. p. 57). Diagnosis of diabetes is by blood tests such as fasting plasma glucose, oral glucose tolerance test, or glycated hemoglobin (A1C) (“Diagnosis of Diabetes and Prediabetes”. National Institute of Diabetes and Digestive and Kidney Diseases. June 2014)

Type 2 diabetes is preventable by staying a normal weight, exercising regularly, and eating a healthy diet (“Diabetes Fact sheet No 312”. World Health Organization. August 2011). If blood sugar levels are not adequately lowered, the medication metformin is typically recommended (Maruthur N M, et al (2016). Annals of Internal Medicine. 164 (11 ): 740-51). Many people may eventually also require insulin injections (Krentz A J et al. (2005). Drugs. 65 (3): 385-411). Bariatric surgery often improves diabetes in those who are obese (Cetinkunar S, et al. (2015). World Journal of Clinical Cases. 3(6 ): 504-9).

Incidence of type 2 diabetes has increased dramatically since 1960 in parallel with obesity. As of 2015 there were approximately 392 million people diagnosed with the disease compared to around 30 million in 1985 (Vos T, et al. (2016). Lancet. 388 (10053 ): 1545-1602). Typically, it begins in middle or older age, although rates of type 2 diabetes are increasing in young people (Imperatore G, et al. (2012). Diabetes Care. 35 (12 ): 2515-20)

As used herein, the term “insulin resistance” is a term related to pathological conditions in which cells fail to respond normally to the hormone insulin (pancreatic hormone associated with glucose homeostasis). There are many causes of insulin resistance and the underlying process is still not completely understood, but high levels of ceramides in tissues is considered as a major determinant of insulin resistance through inhibition cellular insulin signaling cascade (33). Risk factors for insulin resistance include obesity, sedentary lifestyle, family history of diabetes, various health conditions, and certain medications. Insulin resistance is considered a component of the metabolic syndrome. Various genetic factors can increase risk, such as a family history of diabetes, and there are some specific medical conditions associated with insulin resistance, such as polycystic ovary syndrome (Nafiye Y, et al. (2010). Fertility and Sterility. 93 (6 ): 1864-9) and non-alcoholic fatty liver disease (NAFLD). Hepatitis C also makes people three to four times more likely to develop type 2 diabetes and insulin resistance (Milner KL, et al. (2010). Gastroenterology. 138 (3 ): 932-41.e1-3). There are multiple ways to measure insulin resistance such as fasting insulin levels or glucose tolerance tests.

In some embodiments, the prophylactic methods of the invention are particularly suitable for subjects who are identified as at high risk for metabolic disorders. Typically subject that are risk for metabolic disorders include patient with obesity, sedentary lifestyle, family history of diabetes, various health conditions, and certain medications.

Said Bacteroides faecichinchilleae of the present invention can be used as a drug, in particular as a probiotic.

The term “probiotic” has its general meaning in the art and refers to live microorganisms that, when administered in adequate amounts, confer a health benefit on the host” (see: Clinical Infectious Diseases, Volume 46, Issue Supplement_2, 1 Feb. 2008, Pages S58-S61, https://doi.org/10.1086/523341).

It will be understood that the daily dose of the compounds and the composition of the present invention will be decided by the attending physician within the scope of sound medical judgment. The specific therapeutically effective dose for any particular patient will depend upon a variety of factors including the type and severity of the disorder to treat; activity of the specific compound employed; the specific composition employed, the age, body weight, general health, sex and diet of the patient; the time and route of administration and the rate of excretion of the specific compound employed; the duration of the treatment; drugs used in combination or coincidental with the specific bacterium employed and other factors well known in the medical arts. For example, within the skill of the art it is recommended to start the treatment with doses of the compound at levels lower than those required to achieve the desired therapeutic effect and to gradually increase the dosage until the desired effect is achieved.

The compound of the invention may be administered by any suitable route of administration. For example, the compound according to the invention can be administered by oral (including buccal and sublingual), rectal, nasal, topical, pulmonary, vaginal,.

In a preferred embodiment of the invention, the therapeutic composition containing the compound of the invention is administered orally or intrarectally. A rectal administration preferably takes place in the form of a suppository, enema or foam. Intrarectal administration is particularly suitable for intestinal diseases which affect the intestinal sections, for example the gut or in the colon.

### Pharmaceutical Composition

The Bacteroides faecichinchilleae of the present invention, together with one or more conventional adjuvants, carriers, or diluents may be placed into the form of pharmaceutical compositions and unit dosages.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

Accordingly another aspect of the present invention relates to a composition comprising the strain of the invention and in particular a pharmaceutical composition.

The composition, generally defined, is a set of components which is formed at least by the strain of the invention at any concentration.

According to the invention, the previous composition may further comprise at least one additional microorganism other than the strain of the invention and/or its cellular components, metabolites or secreted molecules, or any combination thereof. For example, but not limited to, the additional microorganism that may form part of said composition is selected from among at least one of the following groups:
at least one strain of another species of the genus Bacteroides (such as the species B. uniformis);
at least one lactic acid bacterium or intestinal Bifidobacterium, of alimentary or environmental origin. The lactic bacterium is selected from the list comprising, but not limited to, bacteria of the genus Bifidobacterium, Lactobacillus, Lactococcus, Enterococcus, Propionibacterium, Leuconostoc, Weissella, Pediococcus, or Streptococcus;
at least one strain of other phylogenetic groups, genera or species of intestinal prokaryotes of intestinal, alimentary or environmental origin, such as, but not limited to, Archaea, Firmicutes, Bacteroidetes, Proteobacteria, Actinobacteria, Verrucomicrobia, Fusobacteria, Metanobacteria, Spirochaetes, Fibrobacteres, Deferribacteres, Deinococcus, Thermus, Cyanobacteria, Methanobrevibacterium, Peptostreptococcus, Ruminococcus, Coprococcus, Subdolingranulum, Dorea, Bulleidia, Anaerofustis, Gemella, Roseburia, Catenibacterium, Dialister, Anaerotruncus, Staphylococcus, Micrococcus, Propionibacterium, Enterobacteriaceae, Faecalibacterium, Bacteroides, Parabacteroides, Prevotella, Eubacterium, Akkermansia, Bacillus, Butyrivibrio, or Clostridium;
at least one strain of fungus or yeast such as, but not limited to, belonging to the genus Saccharomyces, Candida, Pichia, Debaryomyces, Torulopsis, Aspergillus, Rhizopus, Mucor or Penicillium.

Said additional microorganism may be a strain of the same species or different species or taxonomic group of microorganisms corresponding to the strain of the invention. The cells comprising the composition may be viable or nonviable, and be in any stage of development or growth (latent, exponential, stationary, etc.), regardless of their morphology.

Preferably, said additional microorganism further comprises at least one intestinal bacterium or one lactic bacterium.

Optionally, the composition according to the present invention further comprises at least one bioactive component (active substance, active ingredient or therapeutic agent) such as, for example, other food, plant and/or pharmaceutical components.

In a preferred embodiment, the composition of the invention is a pharmaceutical composition. The pharmaceutical composition is a set of components which is formed at least by the strain of the invention at any concentration, which implies an improvement in the general state of health or reduced risk of disease. Said pharmaceutical composition can be a drug.

The pharmaceutical composition and unit dosage forms may comprise conventional ingredients in conventional proportions, with or without additional active compounds or principles, and the unit dosage forms may contain any suitable effective amount of the active ingredients commensurate with the intended daily dosage range to be employed. The pharmaceutical composition may be employed as solids, such as tablets or filled capsules, semisolids, powders, sustained release formulations, or liquids such as solutions, suspensions, emulsions, elixirs, or filled capsules for oral use; or in the form of suppositories for rectal administration; or in the form of sterile injectable solutions for parenteral uses. Formulations containing about one (1) milligram of active ingredient or, more broadly, about 0.01 to about one hundred (100) milligrams, per tablet, are accordingly suitable representative unit dosage forms.

The compound of the present invention may be formulated in a wide variety of oral administration dosage forms. The pharmaceutical compositions and dosage forms may comprise compounds of the present invention or pharmaceutically acceptable salts thereof as the active component. The pharmaceutically acceptable carriers may be either solid or liquid. Solid form preparations include powders, tablets, pulls, capsules, cachets, suppositories, and dispersible granules. A solid carrier may be one or more substances which may also act as diluents, flavouring agents, solubilizers, lubricants, suspending agents, binders, preservatives, tablet disintegrating agents, or an encapsulating material. In powders, the carrier generally is a finely divided solid, which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of the active compound. Suitable carriers include but are not limited to magnesium carbonate, magnesium stearate, talc, sugar, lactose, pectin, dextrin, starch gelatin, tragacanth, methylcellulose sodium carboxymethylcellulose, a low melting wax, cocoa butter, and the like.

The term “preparation” is intended to include the formulation of the active compound with an encapsulating material as carrier, providing a capsule in which the active component, with or without carriers, is surrounded by a carrier, which is in association with it. Similarly, cachets and lozenges are included. Tablets, powders, capsules, pulls, cachets, and lozenges may be as solid forms suitable for oral administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions may be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavours, stabilizers, and thickening agents. Aqueous suspensions can be prepared by dispersing the finely divided active component in water with viscous material, such as natural or synthetic gums, resins, methylcellulose, sodium carboxymethylcellulose, and other well-known suspending agents. Solid form preparations include solutions, suspensions, and emulsions, and may contain, in addition to the active component, colorants, flavours, stabilizers, buffers, artificial and natural sweeteners, dispersants, thickeners, solubilising agents, and the like.

Alternatively, the active ingredient may be in powder form, obtained by aseptic isolation of sterile solid or by lyophilization from solution for constitution before use with a suitable vehicle, e.g., sterile, pyrogen-free water.

In another embodiment, when used as probiotic the composition of the present invention suitable for oral administration including powder and liquid form, may be mixed with any food, beverage, cream etc. (such as yogurt or fruit juice).

### Combination of Bacteroides faecichinchilleae and Metformin

As already describe inventors selected Bacteroides faecichinchilleae as the best candidate on the basis of its metformin-mimicking action on gene expression in the duodenum of the treated animals and demonstrated that the strain BAfa have the properties of inhibition of small intestine FXR activity in a preclinical mouse model of diabetes. Inhibition of FXR specifically in the gut has been previously demonstrated as beneficial for metabolic control in diabetic models in rodents (30, 31) and even in type 2 diabetic patients, recent evidence supports a contribution of intestinal FXR inhibition in the beneficial action of metformin (29). Therefore, inventors identified Bacteroides faecichinchilleae, which possesses the ability to improve glucose tolerance in a preclinical model of diabetes, with a unique mechanism of action, closely related to the mechanism of the anti-diabetic drug metformin. This result could open the way for a combination therapy of metformin and BAfa, as this was previously proposed with some Lactobacillus strains (24). The expected advantages of such combination could be a synergistic effect to improve patient health and/or the reduction of the amount of the chemical drug (for example to reduce its well-known side effects (36).

Accordingly, in order to treat or prevent metabolic disorders (such as type 2 diabetes and insulin resistance) means that Bacteroides faecichinchilleae of the invention could be used in combination with metformin.

In some embodiments, the methods and use of the present invention further comprises the step of applying metformin drug.

Accordingly, the present invention also relates to a method of preventing or treating metabolic disorders in a subject comprising administering to the subject a therapeutically effective amount of Bacteroides faecichinchilleae and metformin drug.

The present invention also relates to a pharmaceutical composition comprising a bacteria of the species Bacteroides faecichinchilleae and metformin drug.

The present invention also relates to pharmaceutical composition comprising a bacteria of the species Bacteroides faecichinchilleae and metformin drug for simultaneous or sequential use in preventing or treating metabolic disorder.

In a particular embodiment, the composition may comprise instead of the bacteria of the species Bacteroides faecichinchilleae, cellular components, metabolites, molecules secreted by said bacterial strains and combinations thereof.

The term “metformin” sold under the brand name Glucophage, among others, has its general meaning in the art and refers to the main first-line medication for the treatment of type 2 diabetes, particularly in people who are overweight (Cosentino F, et al. (2020). European Heart Journal. 41 (2): 255-323. ) It is also used in the treatment of polycystic ovary syndrome It is sometimes used as an off-label adjunct to lessen the risk of metabolic syndrome in people who take antipsychotics (de Silva VA, et al (2016). BMC Psychiatry. 16 (1): 341).

Metformin (No CAS: 657-24-9, 1115-70-4 (HCl) ; No CE: 211-517-8; DrugBank: APRD01099) is a biguanide (N, N-dimethylbiguanide see Sirtori C R, et al (1978). Clinical Pharmacology and Therapeutics. 24(6 ): 683-93) antihyperglycemic agent (which works by decreasing glucose production in the liver, by increasing the insulin sensitivity of body tissues, and by increasing GDF15 secretion, which reduces appetite and caloric intake (Coll A P, et al. (2020). Nature. 578(7795 ): 444-448). Metformin was discovered in 1922 and French physician Jean Sterne began the study in humans in the 1950s (Fischer J (2010). Analogue-based Drug Discovery II. John Wiley & Sons. p. 49) It is on the World Health Organization's List of Essential Medicines (World Health Organization (2019). World Health Organization model list of essential medicines: 21st list 2019. Geneva: World Health Organization). Metformin is the most widely used medication for diabetes taken orally (Fischer J (2010). Analogue-based Drug Discovery II. John Wiley & Sons. p. 49) It is available as a generic medication. In 2020, it was the third most-commonly prescribed medication in the United States, with more than 92 million prescriptions.

In other words, the invention relates to a combination of a bacteria of the species Bacteroides faecichinchilleae and metformin drug for the simultaneous or sequential use for preventing or treating metabolic disorder in a subject in need thereof.

In another embodiment, when used as probiotic the composition of the present invention suitable for oral administration including powder and liquid form, may be mixed with any food, beverage, cream etc. (such as yogurt or fruit juice).

By a “therapeutically effective amount” is meant a sufficient amount of compound to treat and/or to prevent metabolic disorder.

It will be understood that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific inhibitor employed; and like factors well known in the medical arts. For example, it is well within the skill of the art to start doses of the compound at levels lower than those required to achieve the desired therapeutic effect and to gradually increase the dosage until the desired effect is achieved.

The combination of Bacteroides faecichinchilleae with metformin according to the invention can be administered by any suitable route of administration. For example, the combination according to the invention can be administered by oral (including buccal and sublingual), rectal, nasal, topical (intravesical), pulmonary administration or in a form suitable for administration by inhalation or insufflation.

In preferred embodiment the combination according to the invention is administered by oral (including buccal and sublingual) or rectal administration.

### FIGURES

FIG. 1. shows that BAfa supplementation by gavage with 10⁹ CFU/day during 8 days restores HFS diet-induced alterations in the level of several genes in the duodenum of the mice, similarly as metformin does. (*p<0.05, **p<0.01, ***p<0.001) and (a p<0.05, b p<0.01, c p<0.005) using post-hoc nonparametric Mann-Withney test

FIG. 2: shows that BAfa treatment during 3 weeks (gavage with 10⁹ CFU/day of live bacteria frozen in a sucrose containing culture medium) significantly improves glucose tolerance in HFS fed mice during iGGT as seen by the comparison of the area under curves (AUC) of the test (a: p=0.0251, with post-hoc Mann Withney test). In the same experiment, live A. muciniphila supplementation (10⁹ CFU/day frozen in glycerol containing medium) also ameliorated glucose tolerance (c: p=0.0031, with post-hoc Mann Withney test).

FIG. 3: shows the effect of BAfa (10⁹ CFU/day by gavage) compared to the effect of metformin (200 mg/kg/day by gavage) on glucose excursion during iGTT. Both treatments significantly improve glucose tolerance after 3 weeks in HFS diet fed mice (a: p=0.0145, with post-hoc Mann Withney test).

FIG. 4: shows the changes in the expression level of FXR target genes in the ileum of the mice after 4 weeks of treatment. BAfa supplementation (10⁹ CFU/day by gavage) significantly down-regulate Fgf15, Shp and Fabp6 gene expression measured by RT-qPCR, while the treatment with A. muciniphila was without effect on these target genes of FXR. (c: p=0.0016, with post-hoc Mann Withney test).

FIG. 5: shows the effects of BAfa and A. muciniphila treatment for 4 weeks (10⁹ CFU/day by gavage) on bile acid abundances in the ceacum of the animals. BAfa clearly reduces the amount of secondary bile acids and especially molecular species that are agonists of FXR. These effects were not observed after A. muciniphila treatment. (a: p<0.05 and b: p<0.01 with post-hoc Mann-Withney test).

FIG. 6: shows that BAfa treatment for 4 weeks (10⁹ CFU/day by gavage) provokes a significant reduction of the concentration of ceramides in the liver of the treated mice. The concentration of C16-ceramide, the most deleterious ceramide molecule regarding insulin action, is reduced by 2-fold in the presence of BAfa.

FIG. 7: Shows that 4 weeks of treatment with BAfa (10⁹ CFU/day by gavage) in HFS fed mice does not significantly reduce body weight gain. However, the treated animals showed a significantly reduced fat mass at the end of the treatment with BAfa (a: p=0.0314, with a post-hoc Mann Withney test).

FIG. 8: Shows a concerted down-regulation of several genes, that are up-regulated in the ileum of HFS fed mice, in response to BAfa treatment during 4 weeks. (*p<0.05, **p<0.01, ***p<0.001).

### EXAMPLE

### Material & Method

Animals, diets and treatments: male C57B1/6 mice of 10 weeks-old were purchased from Envigo and acclimatized 1 week in the animal facility before starting the protocols. Mice were then submitted to a high fat high sucrose (HFS) diet (260HF, SAFE, Augy, France) for either 8 days (screening tests) or 14 weeks (validation experiments in a model of diabetes). In the 8 day-experiments, treatments (with probiotics or metformin) were provided by gavage in a volume of 150 μl, during the 8 days of HFS diet. In the 14 week-experiments, treatments (with probiotics or metformin) were provided by gavage in a volume of 150 μl, starting after 10 weeks of HFS diet and for the following 4 weeks. At the end of the nutritional protocol and the treatments, mice were sacrificed after an overnight fast and tissue (intestinal segments, caecum, liver) were removed and immediately frozen in liquid nitrogen for further use.
Bacteria: The Bacteroides faecichinchilleae strain ST32 was obtained from the German Collection of Microorganisms and Cell Cultures (DSMZ 26883) and cultured under anaerobic condition in dedicated medium adapted from Leedle & Hespell (37). Akkermansia muciniphila was obtained from Prof. Patrice Cani (Louvain Catholic University, Brussels, Belgium).
Glucose tolerance tests (iGTT): After 3 weeks of treatment (one week before the end of the nutritional protocol), iGTT were performed on 6 h-fasted mice. For that, mice were injected ip with glucose (1 mg/g body weight) and blood glucose levels were monitored at T0, T15, T30, T45, T60 and T90 minutes, using a glucometer (drop of blood taken from the end of the tail).
Body composition: Body composition was assessed at week 14 using the Bruker's minispec Whole Body Composition Analyzer based on TD-NMR and providing a precise method for measurement of lean and fat in living mice.
Quantification of gene expression in ileum: Total RNAs from ileum were isolated with TRI-Reagent Solution (Sigma Aldrich). First-strand complementary DNAs were synthesized from 1 μg of total RNAs with TAKARA Prime Script™ RT Reagent kit (TAKARA Bio, Saint-Germain-en-Laye, France). Quantitative PCR assays were carried out using the SYBR® Premix Ex-Taq™ kit (TAKARA Bio) on Rotor-Gene™ 6000 (Corbett Research, Mortlake, Australia) in the presence of specific primers for the genes of interest. The mRNA levels of mouse TATA binding protein (TBP) were used to normalize the data. The sequences of the PCR primers are presented in (38).
Bile acid profiling in caecum: The quantification of the different molecular species and bile acid metabolites was carried out from 100 mg of caecum (frozen and reduced to powder) by HPLC coupled to tandem mass spectrometry (HPLC-MS/MS) (39). The results are expressed in nmol/g of caecum, as shown previously (38).
Ceramide profiling in liver: Concentrations ceramide molecular species were determined according to the method by Kyrklund (40), which was optimized as previously described (41). Briefly, for each sample, total lipids were extracted using 2.5 mL of chloroform/methanol (1:2 v/v) in the presence of 2 deuterium-labelled internal standards (N-heptadecanoyl-D-erythro-sphingosine [C 17:0-Ceramide]; N-palmitoyl[d31]-D-erythro-sphingosylphosphorylcholine [C16:0D31SM] from Avanti Polar Lipids). After 2 hours of shaking and centrifugation (room temperature, 10 minutes, 1900 g), samples were evaporated with liquid nitrogen. The dry samples were dissolved in 1.5 mL of chloroform/methanol (1:2 v/v) and sonicated 30 seconds on ice. Polar lipids were then isolated by saponification and then fractionated and desalted using reverse-phase Bond Elut C18 columns. The final elutions were done with 2×1 mL of chloroform/methanol (12:1 v/v) and 2×1 mL of chloroform/methanol (1:2 v/v) prior to the evaporation of samples with liquid nitrogen. The dry extracts were kept at −20 °C until tandem mass spectrometry analysis (MS/MS). For that, samples were homogenized in 1 mL of chloroform/methanol (1:2 v/v) and analyzed by direct flow injection on a triple-quadrupole mass spectrometer (API 4500 QTRAP MS/MS; Sciex Applied Biosystems) in the positive ionization mode using the multiple reaction monitoring (MRM) method. 12 species of ceramides that are the most abundant and of particular interest in metabolic diseases were quantified (41). These analyses were performed on a MS/MS platform accredited following EN NF ISO 15189 requirements.
Statistics: Results are presented as mean±sem. Statistical analyses were performed using Prism 9. For each parameter, the groups were compared using a Kruskal-Wallis test, followed by a Dunn post-hoc test to characterize the difference between groups. Differences were considered significant when the p value was lower than 0.05 and were represented using stars (* p<0.05, ** p<0.01, *** p<0.001) in the graphs. In some cases, to further highlight the difference between the treatment groups and the HFS control group, a post-hoc nonparametric Mann-Withney test was performed between the two groups. Differences were considered significant when the p value was lower than 0.05 and were represented using letters (a p<0.05, b p<0.01, c p<0.005) in the graphs. Figures were done with GraphPad Prism 9.

### Results

The first step of the research program was the identification of candidate bacteria from available gut metagenomic data from diabetic mice and humans treated with metformin. Our working hypothesized was that metformin should increase the abundance of a number of specific commensal bacteria in the gut, in addition to A. muciniphila(11 ), and that these bacteria may contribute to the beneficial effect of the drug. To identify them, we used metagenomic data (16S sequencing) from the laboratory (ceacum of diabetic mice treated with metformin for 16 weeks) and data from microbiota analyzes in type 2 diabetic patient and diabetic rodents treated with metformin, available in the literature and databases at the time of the analysis in 2017. With all of these data, we carried out a bioinformatic search for commensal species and strains regulated by metformin. To establish the list of candidates, we focused on species/strains whose relative abundance is increased by metformin and which are found in both humans and rodents and, when available, with published data suggesting a link with metabolic health. This work, carried out in 2017-2018, enabled us to establish a list of 28 candidate strains/species.

Then we used an original screening test in mice to qualify the antidiabetic potential of some of these candidates (ie the bacteria that were available in biobanks and/or collections). The screening test consisted in the direct comparison of the effect of the candidate bacterium with the effect of metformin (both provided by gavage) during 8 days in mice fed a HFS diet. We indeed previously showed and published (24) that 8 days of HFS nutritional challenge led to metabolic alterations, associated with significant changes in the expression of key genes in different segments of the intestine (glucose transporters in the duodenum, lipid transporters in the jejunum, bile acid signaling in the ileum and inflammation-related gene in the colon) and, importantly, that the expression of these genes was completely restored when the animals were treated with metformin (300 mg/kg/day by oral gavage) during the 8 days of HFS diet (24). We therefore hypothesized that if metformin action is mediated by changes in the abundance of specific bacteria in the gut, strains with antidiabetic properties should be able to reproduce all or part of the effects of metformin on intestinal gene expression. This method therefore makes it possible to screen and qualify bacteria in few days, in vivo in a relevant animal model of metabolic diseases.

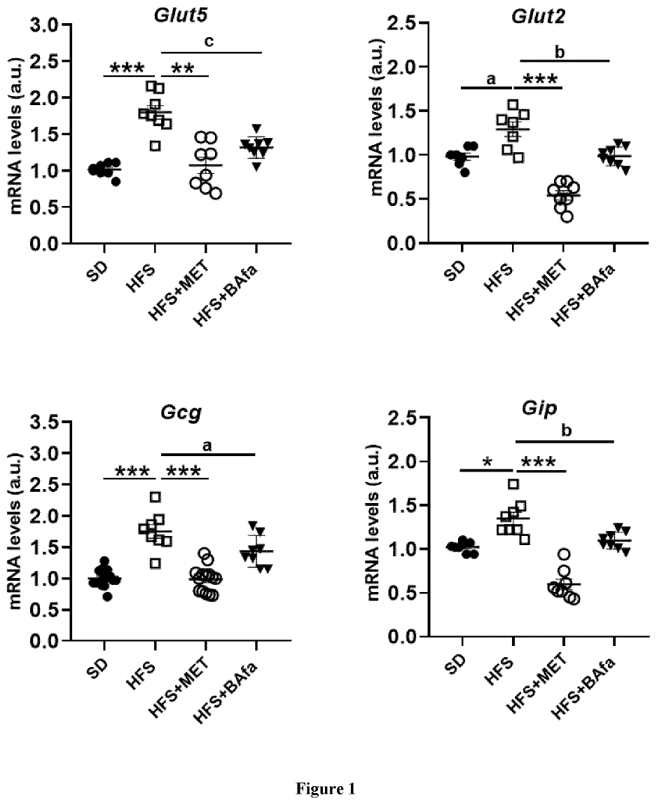

Among the tested candidates (which included several Lactobacillus of the species acidophilus, intestinalis, rhamnosus, johnsonii as well as Christensenella minuta) and some additional bacteria of potential interest such as Lactobacillus reuteri, Faecalibacterium Prausnitzii and Akkermansia muciniphila, which were not in the candidate list, we identified Bacteroides faecichinchilleae (ST37 strain further named BAfa for simplification) as a commensal bacterium able to reproduce the effect of metformin of the expression of the glucose transporter (Glut2), the fructose transporter (Glut5), the glucagon (Gcg) and the gastric inhibitory polypeptide (Gip) genes, in the duodenum of the HFS-fed mice (FIG. 1). These results strongly suggested that BAfa may potentially have beneficial effects on glucose/fructose absorption and insulin regulation, important hallmarks of type 2 diabetes. Further supporting a beneficial role of BAfa in metabolic disorders, the abundance of a Bacteroides faecichinchilleae strain was previously found significantly decreased in the gut microbiota of obese compared to healthy lean individuals in the Japanese population (42).

We further investigated the potential of BAfa in the context of diabetes by testing its capacity to improve glucose tolerance in mice submitted to HFS diet for longer duration. Classically, 8 to 10 weeks of high calorie diet are needed to produce metabolic alterations, especially decreased glucose tolerance and moderate increase in glycemia and insulinemia. More marked defects (liver fat accumulation, insulin resistance or inflammation) are generally observed after 14/16 weeks of diet (43). To verify possible beneficial effects of BAfa, we decided to perform a “treatment” protocol, consisting in a supplementation with the bacteria preparation during 4 weeks in mice maintained on HFS diet and that have been fed already with the same diet for 10 weeks. In addition, the effects of BAfa (1.0×10⁹ live bacteria per day and per mouse) were compared to a similar treatment (daily gavage) with Akkermansia muciniphila (provided by Prof. P. Cani, Brussels), as a validated reference of “next generation” probiotics, or with metformin (200 mg/kg/day).

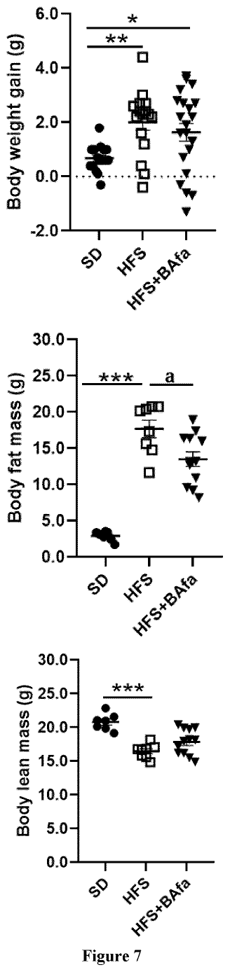

After 3 weeks of treatment, intraperitoneal glucose tolerance tests (iGTT) were performed as the reference method to evaluate glucose tolerance in vivo. One week after (i.e. after 4 weeks of treatment and 14 weeks of HFHS diet), mice were sacrificed and tissues were collected for molecular and histological analyses. Evolution of weight during the protocol was followed by weekly individual measurements of body weight, difference in body composition was assessed at the end of the treatment using a Bruker's minispec LF-50, and food consumption was estimated by the quantification of remaining food in mouse cages. As shown in FIG. 7, live BAfa did not significantly affect animal body weight and body composition after 4 weeks of treatment, although there was a tendency for a slight reduction of body fat (and body weight), that will require further investigation. Food consumption was not modified by BAfa supplementation (not shown).

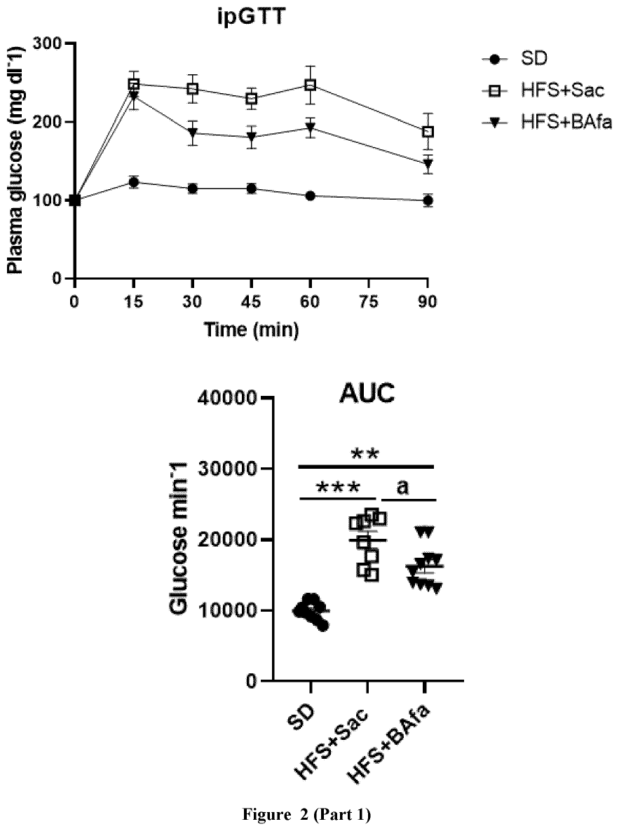

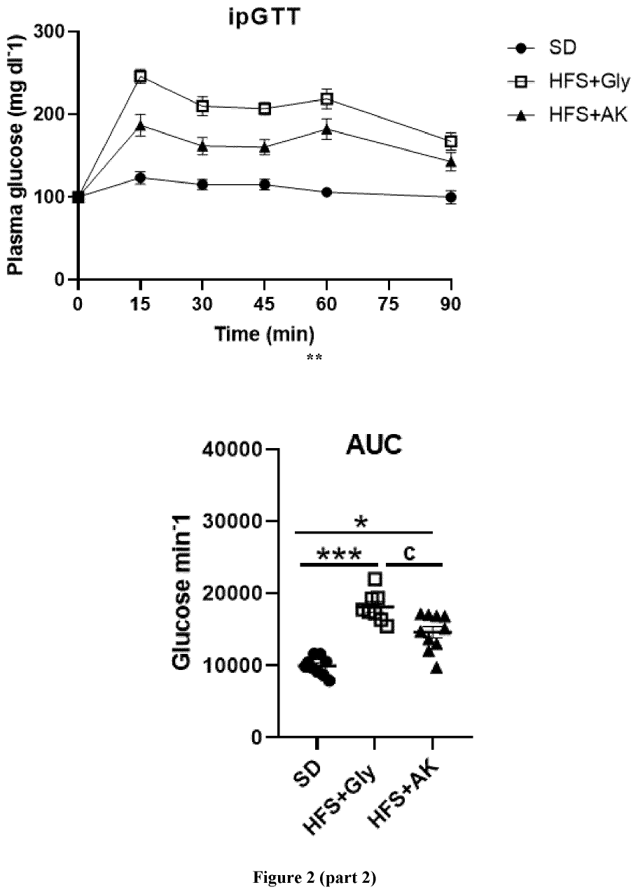

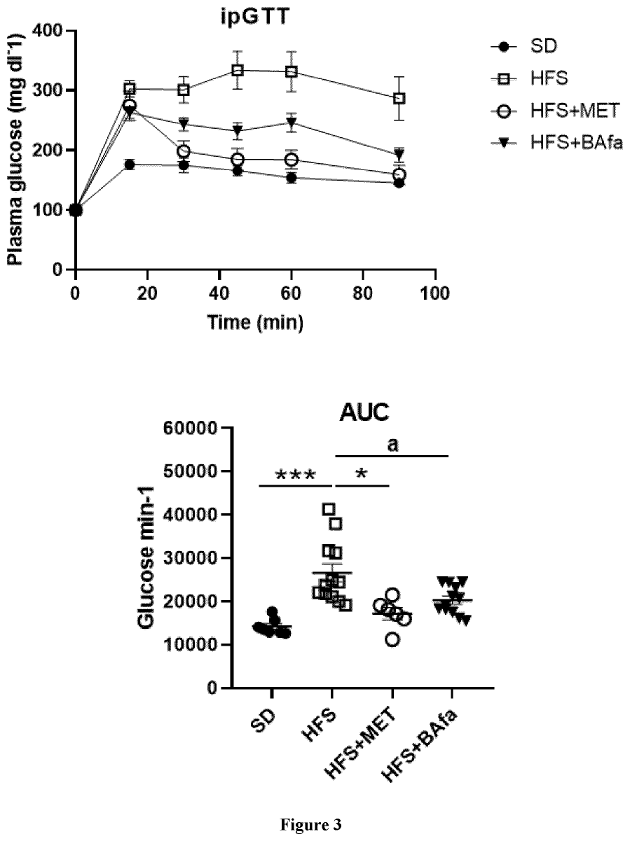

Two distinct experiments were performed, with 10 to 15 mice per groups and with separated preparations of BAfa. The bacteria were cultivated under anaerobic conditions and frozen at the required concentration (1.0×10⁹ CFU in 150 μl) after adding a cryoprotectant medium containing sucrose, and aliquoted in glass tubes deprived of oxygen. We verified that the procedure and the storage at −80 °C did not affect viability. We found that treatment of HFS-fed mice for 3 weeks with live BAfa (10⁹ cfu/day) significantly improved glucose tolerance during intraperitoneal glucose tolerance tests (iGTT), with effects comparable to Akkermansia muciniphila supplementation (10⁹ cfu/day) (FIG. 2). Although the effect of BAfa was less important than the effect of metformin used at maximal dosage (200 mg/kg/day, for 3 weeks) (FIG. 3), the results of these experiments (FIGS. 2 and 3) clearly demonstrate that BAfa supplementation improves glucose tolerance in a preclinical model of diabetes.

To further decipher the possible mechanisms of action contributing to the effect of BAfa on glucose tolerance, we investigated the involvement of different metabolic and signaling pathways in tissues that were sampled at the end of the protocols.

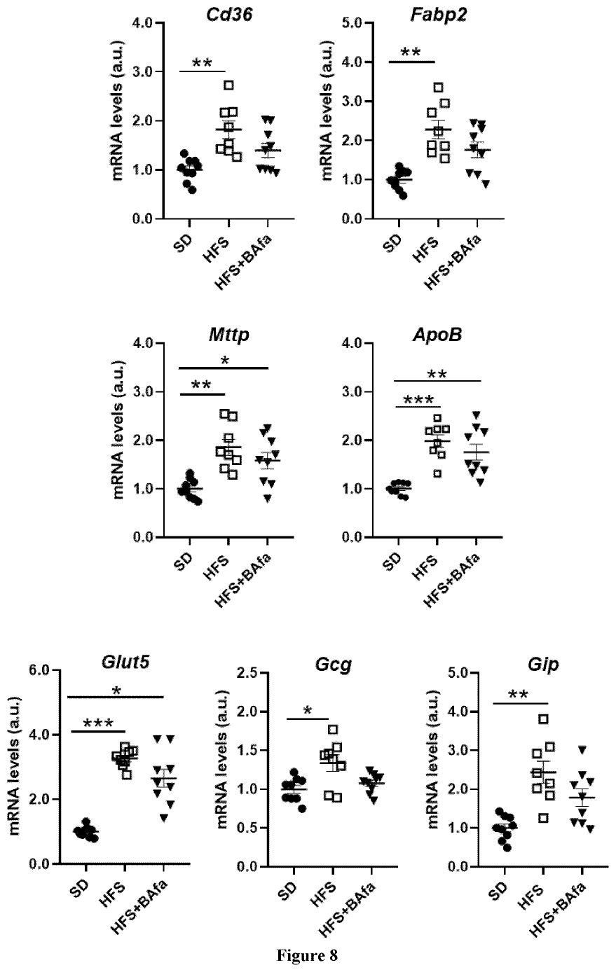

The first interesting observation was a concerted reduction in the expression levels of genes coding key actors of nutrient absorption in the ileum of the mice. As shown in FIG. 8, HFS diet was associated with a significant increase in the gene expression of Cd36 (fatty acid transporter), Fabp2 (intracellular fatty acid binding protein), Mttp and ApoB (2 major regulators of chylomicron production and lipid absorption) and of Glut5 (fructose transporter). For all these genes, BAfa appeared to counteract the effect of HFS diet. Although not significant due to the low number of samples, this concerted effect on a network of genes suggests that BAfa treatment could be associated with a reduction of fructose and lipid absorption. Then, and also consistent with the observation made initially during short term (8 days) protocols, we found that BAfa supplementation for 4 weeks in HFS diet mice was associated with a reduction in the expression of the genes coding the incretin hormones GLP1 (Gcg) and GIP (Gip) in the ileum (FIG. 8).

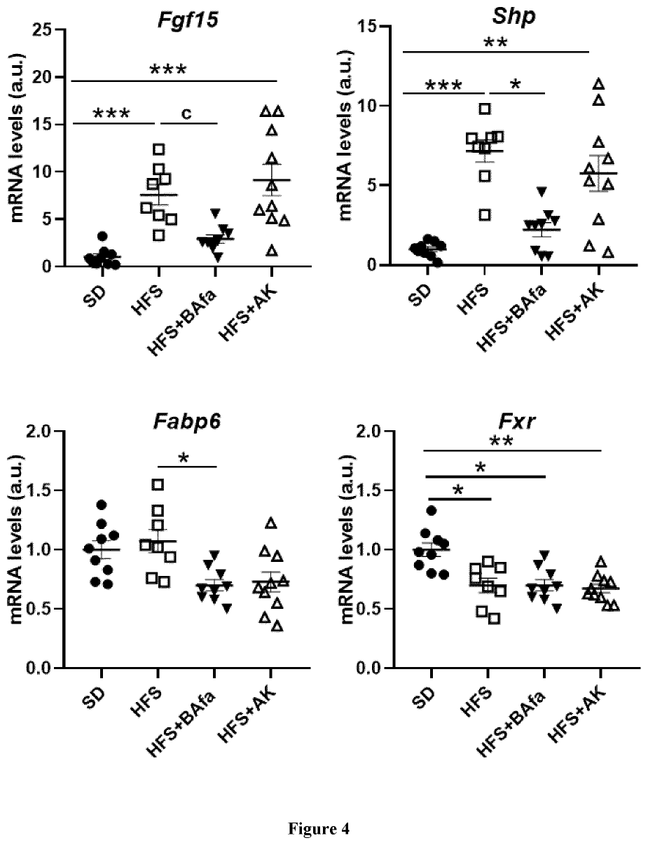

To further understand the mechanism involved in BAfa beneficial effects, we evaluated the impact of the treatment on FXR/FGF15 pathway in the ileum. The farnesoid X receptor (FXR), plays a pivotal role in regulating bile acid, lipid and glucose metabolism, but also inflammation and intestinal barrier function (25). In the ileum, FXR controls the gene expression of Fgf15 (fibroblast growth factor 15 in rodent and FGF19 in humans), which recently emerged as an important regulator of glucose and lipid metabolism in addition to its role in the control of bile acid synthesis in the liver (26, 27). We found that supplementation of HFS mice with BAfa, but not with A. muciniphila, resulted in a strong inhibition of the expression of the target genes of FXR in the ileum, namely Fgf15, Shp/Nrob2 (small heterodimer partner 1) and Fabp6 (Fatty acid binding protein 6, coding for the ileal intracellular bile acid transporter). The expression of these 3 genes was increased by HFD diet and markedly reduced after 4 weeks of supplementation with BAfa (FIG. 4). The expression of Fxr itself was decreased by the HFS diet, but not affected by BAfa. These results indicated thus that BAfa supplementation is associated with an inhibition of FXR signaling in the ileum. It was recently reported that inhibition of intestinal FXR activity exerts potent anti-diabetic effects (30, 31). BAfa beneficial metabolic action could therefore be the results of an inhibition of FXR in the intestinal tract. Importantly, this mechanism is specific of BAfa since it was not observed in the presence of A. Muciniphila (FIG. 4).

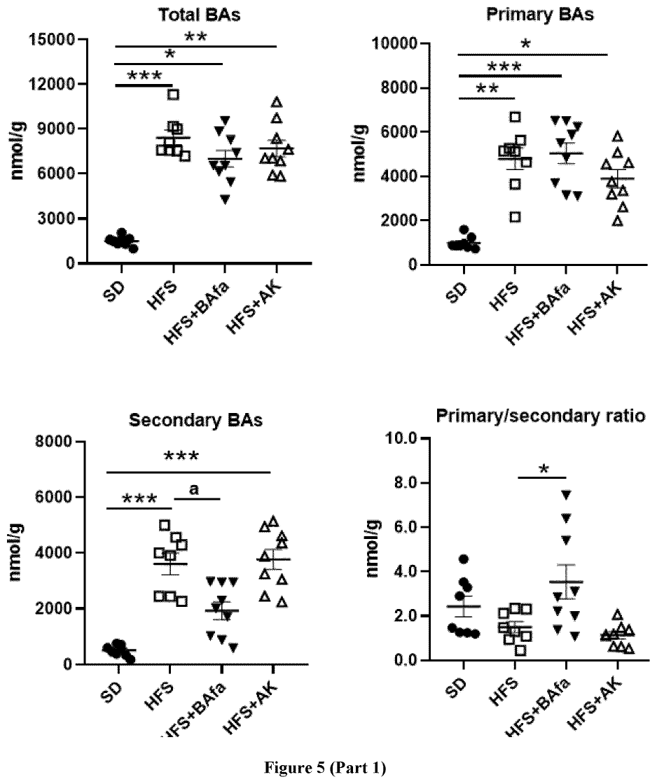

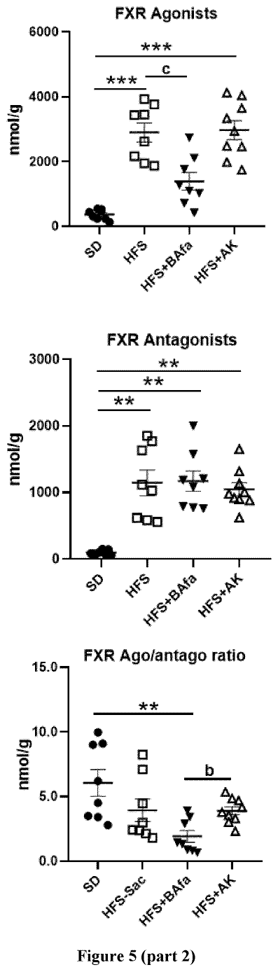

To identify the causes of FXR inhibition in the presence of BAfa, bile acid profiling was performed in the ceacum of the treated mice. Bile acids are natural ligands of FXR and can be either activators or inhibitors, with CDCA (chenodeoxycholic acid) and DCA (deoxycholic acid) being the more potent agonists, whereas UDCA (ursodeoxycholic acid) and B-MCA (muricholic acid) are antagonists of FXR (28, 31). FIG. 5 shows that BAfa supplementation, and not A. muciniphila, changed the primary to secondary bile acid ratio by decreasing the amount of secondary bile acids. Then, when the sum of the bile acids able to stimulate FXR or to inhibit FXR (UDCA/TUDCA/B-MCA/TMCA) was calculated, it was clear that the treatment with BAfa, and not with A. muciniphila, was associated with a significant reduction in the amount of FXR agonists and thus a reduction in the ratio between agonists and antagonists (FIG. 5), likely contributing to the observed inhibition of FXR activity in the ileum.

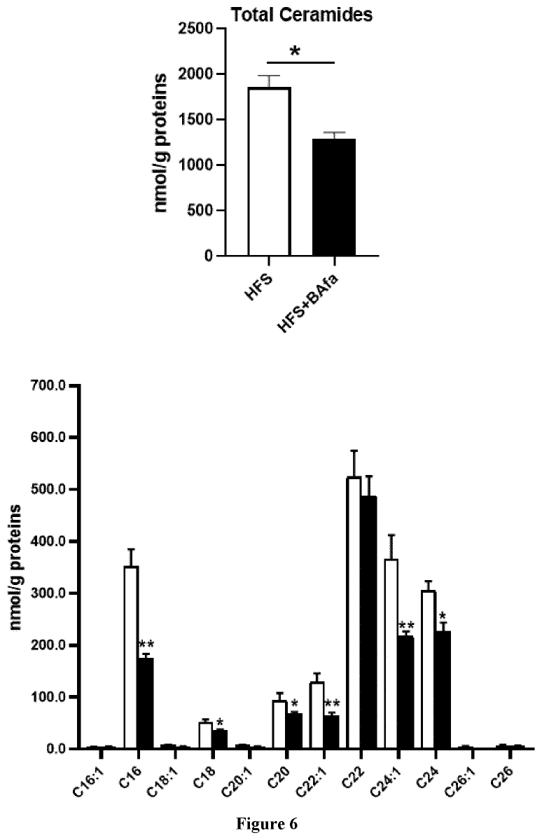

Importantly, the inhibition of FXR activity in the small intestine has been associated with an improvement of metabolic health in animal models (30, 31) and recently, the effect of metformin in type 2 diabetic patients has been also related to intestinal FXR inhibition in response to bile acid changes (29). We also observed previously similar mechanism in diabetic mice treated with metformin (38). A proposed explanation for the beneficial effect of FXR inhibition in the intestinal tract is the regulation of the production of ceramides and their accumulation in the liver (30, 31). Ceramides are major contributors to insulin resistance, inflammation and metabolic defects and are directly implicated in the inhibition of insulin signaling (32, 33). We therefore quantified the different ceramides species in the liver of the treated mice. FIG. 6 clearly shown that 4 weeks treatment with BAfa was able to significantly reduce the amount of various ceramide species in the liver of the HFS fed mice, especially the C16- and C18-ceramides that are the most deleterious species of ceramides regarding insulin action and risk of type 2 diabetes (33).

In conclusion, we identified a novel species of commensal bacteria, Bacteroides faecichinchilleae, which possesses the ability to improve glucose tolerance in a preclinical model of diabetes, with a unique mechanism of action, closely related to the mechanism of anti-diabetic drug metformin. We propose therefore that BAfa and the related strains of Bacteroides faecichinchilleae, could be considered for the development of next-generation probiotics for the treatment of type 2 diabetes, optimally in combination with classical anti-diabetic drug such as metformin.

### REFERENCES

Throughout this application, various references describe the state of the art to which this invention pertains. The disclosures of these references are hereby incorporated by reference into the present disclosure.
1- WHO-Global status report on noncommunicable diseases 2014
2- Cani P D. Metabolism in 2013: The gut microbiota manages host metabolism. Nat Rev Endocrinol. 2014,10:74-6.
3- Ley R E, Turnbaugh P J, Klein S, Gordon J I. Microbial ecology: human gut microbes associated with obesity. Nature. 2006 December 21; 444(7122):1022-3
4- Qin J, Li Y, Cai Z, Li S, Zhu J, Zhang F, Liang S, et al. A metagenome-wide association study of gut microbiota in type 2 diabetes. Nature. 2012 October 4; 490(7418):55-60.
5- Karlsson F H, Tremaroli V, Nookaew I, Bergström G, Behre C J, Fagerberg B, Nielsen J, Bäckhed F. Gut metagenome in European women with normal, impaired and diabetic glucose control. Nature. 2013 June 6; 498(7452):99-103.
6- Gérard C, Vidal H. Impact of Gut Microbiota on Host Glycemic Control. Front Endocrinol (Lausanne). 2019 January 30; 10:29.
7- Turnbaugh P J, Ley R E, Mahowald M A, Magrini V, Mardis E R, Gordon J I. An obesity-associated gut microbiome with increased capacity for energy harvest. Nature. 2006 December 21; 444(7122):1027-31.
8- Wong S K, Chin K Y, Suhaimi F H, Fairus A, Ima-Nirwana S. Animal models of metabolic syndrome: a review. Nutr Metab (Lond). 2016 October 4; 13:65.
9- Turnbaugh P J, Bäckhed F, Fulton L, Gordon J I. Diet-induced obesity is linked to marked but reversible alterations in the mouse distal gut microbiome. Cell Host Microbe. 2008 April 17; 3(4):213-23.
10- Wu H, Esteve E, Tremaroli V, Khan M T, Caesar R, Mannerås-Holm L, Ståhlman M, Olsson L M, Serino M, Planas-Felix M, Xifra G, Mercader J M, Torrents D, Burcelin R, Ricart W, Perkins R, Fernandez-Real J M, Bäckhed F. Metformin alters the gut microbiome of individuals with treatment-naive type 2 diabetes, contributing to the therapeutic effects of the drug. Nat Med. 2017 Jul; 23:850-858.
11- Shin N R, Lee J C, Lee H Y, Kim M S, Whon T W, Lee M S, Bae J W. An increase in the Akkermansia spp. population induced by metformin treatment improves glucose homeostasis in diet-induced obese mice. Gut. 2014 May; 63(5):727-35.
12- Hsieh F C, Lee C L, Chai C Y, Chen W T, Lu Y C, Wu C S. Oral administration of Lactobacillus reuteri GMNL-263 improves insulin resistance and ameliorates hepatic steatosis in high fructose-fed rats. Nutr Metab (Lond). 2013 April 17; 10(1):35.
13- Won S M, Chen S, Lee S Y, Lee K E, Park K W, Yoon J H. Lactobacillus sakei ADM14 Induces Anti-Obesity Effects and Changes in Gut Microbiome in High-Fat Diet-Induced Obese Mice. Nutrients. 2020 November 30; 12(12):3703.
14- da Costa W K A, Brandão L R, Martino M E, Garcia E F, Alves A F, de Souza E L, de Souza Aquino J, Saarela M, Leulier F, Vidal H, Magnani M. Qualification of tropical fruit-derived Lactobacillus plantarum strains as potential probiotics acting on blood glucose and total cholesterol levels in Wistar rats. Food Res Int. 2019 Oct; 124:109-117.
15- Alard J, Cudennec B, Boutillier D, Peucelle V, Descat A, Decoin R, Kuylle S, Jablaoui A, Rhimi M, Wolowczuk I, Pot B, Tailleux A, Maguin E, Holowacz S, Grangette C. Multiple Selection Criteria for Probiotic Strains with High Potential for Obesity Management. Nutrients. 2021 February 24; 13(3):713
16- Le Barz M, Daniel N, Varin T V, Naimi S, Demers-Mathieu V, Pilon G, Audy J, Laurin É, Roy D, Urdaci M C, St-Gelais D, Fliss I, Marette A. In vivo screening of multiple bacterial strains identifies Lactobacillus rhamnosus Lb102 and Bifidobacterium animalis ssp. lactis Bf141 as probiotics that improve metabolic disorders in a mouse model of obesity. FASEB J. 2019 Apr; 33(4):4921-4935.
17- Andreasen A S, Larsen N, Pedersen-Skovsgaard T, Berg R M, Møller K, Svendsen K D, Jakobsen M, Pedersen B K. Effects of Lactobacillus acidophilus NCFM on insulin sensitivity and the systemic inflammatory response in human subjects. Br J Nutr. 2010 Dec; 104(12):1831-8.
18- Hulston C J, Churnside A A, Venables M C. Probiotic supplementation prevents high-fat, overfeeding-induced insulin resistance in human subjects. Br J Nutr. 2015 February 28; 113(4):596-602.
19- Ruan Y, Sun J, He J, Chen F, Chen R, Chen H. Effect of probiotics on glycemic control: a systematic review and meta-analysis of randomized, controlled trials. PLOS One. 2015 July 10; 10(7):e0132121
20- Yao K, Zeng L, He Q, Wang W, Lei J, Zou X. Effect of Probiotics on Glucose and Lipid Metabolism in Type 2 Diabetes Mellitus: A Meta-Analysis of 12 Randomized Controlled Trials. Med Sci Monit Int Med J Exp Clin Res. 22 juin 2017; 23:3044-53.
21- Plovier H, Everard A, Druart C, Depommier C, Van Hul M, Geurts L, Chilloux J, Ottman N, Duparc T, Lichtenstein L, Myridakis A, Delzenne N M, Klievink J, Bhattacharjee A, van der Ark K C, Aalvink S, Martinez L O, Dumas M E, Maiter D, Loumaye A, Hermans M P, Thissen J P, Belzer C, de Vos WM, Cani P D. A purified membrane protein from Akkermansia muciniphila or the pasteurized bacterium improves metabolism in obese and diabetic mice. Nat Med. 2017 Jan; 23(1):107-113.
22- Depommier C, Everard A, Druart C, Plovier H, Van Hul M, Vieira-Silva S, et al. Supplementation with Akkermansia muciniphila in overweight and obese human volunteers: a proof-of-concept exploratory study. Nat Med. 2019; 25(7):1096-103.
23- Chang C-J, Lin T-L, Tsai Y-L, Wu T-R, Lai W-F, Lu C-C, et al. Next generation probiotics in disease amelioration. J Food Drug Anal. juill 2019; 27(3):615-22.
24- Seicaru E M, Popa Ilie I R, Cătinean A, Crăciun A M, Ghervan C. Enhancing Metformin Effects by Adding Gut Microbiota Modulators to Ameliorate the Metabolic Status of Obese, Insulin-Resistant Hosts. J Gastrointestin Liver Dis. 2022 September 16; 31(3):344-354.
25- Sun L, Cai J, Gonzalez F J. The role of farnesoid X receptor in metabolic diseases, and gastrointestinal and liver cancer. Nat Rev Gastroenterol Hepatol. 2021 May; 18(5):335-347.
26- Kir S, Beddow S A, Samuel V T, Miller P, Previs S F, Suino-Powell K, Xu H E, Shulman G I, Kliewer S A, Mangelsdorf D J. FGF19 as a postprandial, insulin-independent activator of hepatic protein and glycogen synthesis. Science. 2011 March 25; 331(6024):1621-4
27- Benoit B, Meugnier E, Castelli M, Chanon S, Vieille-Marchiset A, Durand C, Bendridi N, Pesenti S, Monternier P A, Durieux A C, Freyssenet D, Rieusset J, Lefai E, Vidal H, Ruzzin J. Fibroblast growth factor 19 regulates skeletal muscle mass and ameliorates muscle wasting in mice. Nat Med. 2017 Aug; 23(8):990-996
28- Kiriyama Y, Nochi H. Physiological Role of Bile Acids Modified by the Gut Microbiome. Microorganisms. 2021 December 30; 10(1):68. doi: 10.3390/microorganisms 10010068.
29- Sun L, Xie C, Wang G, Wu Y, Wu Q, Wang X, Liu J, Deng Y, Xia J, Chen B, Zhang S, Yun C, Lian G, Zhang X, Zhang H, Bisson W H, Shi J, Gao X, Ge P, Liu C, Krausz K W, Nichols R G, Cai J, Rimal B, Patterson A D, Wang X, Gonzalez F J, Jiang C. Gut microbiota and intestinal FXR mediate the clinical benefits of metformin. Nat Med. 2018 Dec; 24(12):1919-1929
30- Xie C, Jiang C, Shi J, Gao X, Sun D, Sun L, Wang T, Takahashi S, Anitha M, Krausz KW, Patterson AD, Gonzalez FJ. An Intestinal Farnesoid X Receptor-Ceramide Signaling Axis Modulates Hepatic Gluconeogenesis in Mice. Diabetes. 2017 Mar; 66(3):613-626.
31- Gonzalez F J, Jiang C, Patterson A D. An Intestinal Microbiota-Farnesoid X Receptor Axis Modulates Metabolic Disease. 2016 Nov; 151(5):845-859. doi:
10.1053/j.gastro.2016.08.057
32- Chaurasia B, Summers S A. Ceramides-Lipotoxic Inducers of Metabolic Disorders. Trends Endocrinol Metab. 2015 Oct; 26(10): 538-550
33- Sokolowska E, Blachnio-Zabielska A. The Role of Ceramides in Insulin Resistance. Front Endocrinol (Lausanne). 2019 August 21; 10:577. doi: 10.3389/fendo.2019.00577.
34- Li Y, Talbot C L, Chandravanshi B, Ksiazek A, Sood A, Chowdhury K H, Maschek J A, Cox J, Babu A K S, Paz H A, Babu P V A, Meyerholz D K, Wankhade U D, Holland W, Shyong Tai E, Summers S A, Chaurasia B. Cordyceps inhibits ceramide biosynthesis and improves insulin resistance and hepatic steatosis. Sci Rep. 2022 May 4; 12(1):7273. doi: 10.1038/s41598-022-11219-3.
35- Gauffin Cano P, Santacruz A, Moya A, Sanz Y. Bacteroides uniformis CECT 7771 ameliorates metabolic and immunological dysfunction in mice with high-fat-diet induced obesity. PLOS One. 2012; 7(7): e41079.
36-Davidson MB, Peters AL. An overview of metformin in the treatment of type 2 diabetes mellitus. Am J Med. 1997 Jan; 102(1):99-110.
37- Leedle J A Z & Hespell R B (1980). Differential carbohydrate media and anaerobic replica plating techniques in delineating carbohydrate utilizing subgroups in rumen bacterial populations. Applied and Environmental Microbiology, 39, 709-719.
38- Bravard A, Gérard C, Defois C, Benoit B, Makki K, Meugnier E, Rainteau D, Rieusset J, Godet M, Vidal H. Metformin treatment for 8 days impacts multiple intestinal parameters in high-fat high-sucrose fed mice. Sci Rep. 2021 August 17; 11(1):16684
39- Humbert L, Maubert M A, Wolf C, Duboc H, Mahé M, Farabos D, Seksik P, Mallet J M, Trugnan G, Masliah J, Rainteau D. 2012. Bile acid profiling in human biological samples: comparison of extraction procedures and application to normal and cholestatic patients. J. Chromatogr. B Analyt. Technol. Biomed. Life. Sci. 899, 135-145. https://doi.org/10.1016/j.jchromb.2012.05.015
40- Kyrklund T. Two procedures to remove polar contaminants from a crude brain lipid extract by using prepacked reversed-phase columns. Lipids. 1987 Apr; 22(4):274-7. doi: 10.1007/BF02533991.
41- Le Barz M, Vors C, Combe E, Joumard-Cubizolles L, Lecomte M, Joffre F, Trauchessec M, Pesenti S, Loizon E, Breyton A E, Meugnier E, Bertrand K, Drai J, Robert C, Durand A, Cuerq C, Gaborit P, Leconte N, Bernalier-Donadille A, Cotte E, Laville M, Lambert-Porcheron S, Ouchchane L, Vidal H, Malpuech-Brugere C, Cheillan D, Michalski M C. Milk polar lipids favorably alter circulating and intestinal ceramide and sphingomyelin species in postmenopausal women. JCI Insight. 2021 May 24; 6(10): e146161. doi: 10.1172/jci.insight.146161
42- Kasai C, Sugimoto K, Moritani I, Tanaka J, Oya Y, Inoue H, Tameda M, Shiraki K, Ito M, Takei Y, Takase K. Comparison of the gut microbiota composition between obese and non-obese individuals in a Japanese population, as analyzed by terminal restriction fragment length polymorphism and next-generation sequencing. BMC Gastroenterol. 2015 August 11; 15:100.
43- Beaulant A, Dia M, Pillot B, Chauvin M A, Ji-Cao J, Durand C, Bendridi N, Chanon S, Vieille-Marchiset A, Da Silva C C, Patouraux S, Anty R, Iannelli A, Tran A, Gual P, Vidal H, Gomez L, Paillard M, Rieusset J. Endoplasmic reticulum-mitochondria miscommunication is an early and causal trigger of hepatic insulin resistance and steatosis. J Hepatol. 2022 Sep; 77(3):710-722.

1. A method of preventing or treating a metabolic disorder in a subject in need thereof comprising administering to the subject a therapeutically effective amount of a bacteria of the species Bacteroides faecichinchilleae.

2. (canceled)

3. The method of claim 1, wherein the metabolic disorder is selected from the group consisting of excess weight, obesity, hepatic steatosis, of fatty liver, dyslipidemia hyperglycemia, insulin resistance, diabetes, metabolic syndrome, hypertension and a cardiovascular diseases.

4. The method according to claim 3, wherein the metabolic disorder is Type 2 diabetes mellitus or insulin resistance.

5. The method according to claim 1, wherein said Bacteroides faecichinchilleae improves glucose tolerance and/or inhibits intestinal FXR activity and/or reduces liver ceramide concentrations.

6. A pharmaceutical composition comprising a bacteria of the species Bacteroides faecichinchilleae and metformin.

7. (canceled)

8. The method of claim 1 further comprising administering to the subject a therapeutically effective amount of metformin.

9. (canceled)

10. The method of claim 3, wherein the dyslipidemia is hypercholesterolemia and/or hypertriglyceridemia.

11. The method of claim 3, wherein the diabetes is Type 2 diabetes mellitus or gestational diabetes.

12. The method of claim 8, wherein the therapeutically effective amount of the bacteria of the species Bacteroides faecichinchilleae and the metformin are administered simultaneously or sequentially.

13. The method of claim 1, wherein the bacteria of the species Bacteroides faecichinchilleae is administered as a drug to treat the metabolic disorder and/or as a probiotic to prevent the metabolic disorder.
